(19) United States
(12) Patent Application Publication
(10) Pub. No.: US 2011/0082454 A1
(43) Pub. Date: Apr. 7, 2011

(54) Cryosurgical System

(75) Inventors: Russell L. DeLonzor, Pleasanton, CA (US); James B. Ross, Pleasanton, CA (US); Mathew J. Nalipinski, Pleasanton, CA (US); Keith Turner, Pleasanton, CA (US); David J. Foster, Cambridge (GB); Michael R. Cane, Cambridge (GB); Samuel C. Richards, Cambridge (GB); David J. Selvey, Cambridge (GB)

(73) Assignee: Sanarus Technologies, LLC

(21) Appl. No.: 12/898,238

(22) Filed: Oct. 5, 2010

Related U.S. Application Data
(63) Continuation of application No. 12/407,303, filed on Mar. 19, 2009, now abandoned.
Application No. 12/407,303 is a continuation of application No. 11/406,547, filed on Apr. 18, 2006, now abandoned.

(51) Int. Cl. A61B 18/02

(52) U.S. Cl. 606/23

(57) ABSTRACT

A cryosurgical system using a low-pressure liquid nitrogen supply, which requires only 0.5 to 15 bar of pressure to provide adequate cooling power for treatment of typical breast lesions. The pressure may be provided by supplying lightly pressurized air into the dewar, by heating a small portion of the nitrogen in the dewar, or with a small low pressure pump.

This application is a continuation application of which is a continuation application of Ser. No. 12/407,303 filed Mar. 19, 2009, which is a continuation of application Ser. No. 11/406,547, filed Apr. 18, 2006.

### FIELD OF THE INVENTIONS

The inventions described below relate the field of cryosurgical systems.

### BACKGROUND OF THE INVENTIONS

Cryosurgery refers to the freezing of body tissue in order to destroy diseased tissue. Minimally invasive cryosurgical systems generally include a long, slender cryoprobe adapted for insertion into the body so that the tip resides in the diseased tissue, and source of cryogenic fluid, and the necessary tubing to conduct the cryogenic fluid into and out of the probe. These cryosurgical systems also include heating systems, so that the probes can be warmed to enhance the destructive effect of the cryoablation and to provide for quick release of the cryoprobes when ablation is complete.

Our own Visica® cryoablation system has proven effective for the treatment of lesions within the breast of female patients. The system uses Joule-Thompson cryoprobes, and uses argon gas as the cryogenic fluid. The argon gas, supplied at room temperature but very high pressure, expands and cools within the tip of the cryoprobe to generate the cooling power needed to freeze body tissue to cryogenic temperatures. The Visica® cryoablation system uses high-pressure helium flow through the cryoprobe to heat the probe. The system requires large supplies of argon gas, but is otherwise quite convenient.

Present cryoprobes utilizing Joule-Thomson systems have inherent disadvantages such as inefficient heat transfer and excessive use of cryogen. As a result, these systems require large quantities of gasses under high pressure and high flow rates. Use of high-pressure gasses increases the overall costs of cryoprobes. This is due to the high cost of materials required for use with systems utilizing high-pressure gases, the high costs associated with obtainib high pressure gases and the large quantities of cryogen required for use with these systems.

Earlier cryoprobes proposed for other surgeries, such as prostrate cryosurgery, used liquid nitrogen, which has the advantage that it is more readily available than argon, and the volume necessary for a given cryosurgical procedure is much smaller then argon. Cryoablation systems using liquid nitrogen, such as the Accuprobe™ cryoablation system, have been proposed and used, but these systems have been abandoned in favor of the Joule-Thompson systems. The literature and patent filings indicate that liquid nitrogen systems were plagued by various problems, such as vapor lock and excessive consumption of liquid nitrogen. Proposals to solve these problems, though never successfully implemented, include various schemes to prevent vapor lock and maximize efficiency of the heat exchange. See Rubinsky, et al., Cryosurgical System For Destroying Tumors By Freezing, U.S. Pat. No. 5,334,181 (Aug. 2, 1994) and Rubinsky, et al., Cryosurgical Instrument And System And Method Of Cryosurgery, U.S. Pat. No. 5,674,218 (Oct. 7, 1997), and Littrup, et al., Cryotherapy Probe and System, PCT Pub. WO 2004/064914 (Aug. 5, 2004). Systems like those disclosed in Rubinsky '181, Rubinski '218 and Littrup are complicated and expensive to manufacture.

Rubinsky '181 and '218 are extremely complex systems. The Rubinsky system is directed towards a system that includes a vacuum chamber and means for drawing a vacuum on a reservoir of liquid nitrogen while sub-cooling the liquid nitrogen. Specifically, the system accomplishes the sub-cooling of liquid nitrogen by evaporative cooling induced by using an active vacuum on a reservoir of liquid nitrogen. The liquid nitrogen (LN₂) in Rubinsky flows through a heat exchanger disposed within a vacuum chamber prior to entering the probe through an inlet tube. The LN₂ is sub-cooled to temperatures far below −195.8° C. (sub-cooling) in the vacuum chamber.

Rubinsky takes the drastic approach of sub-cooling the LN₂ in an effort to overcome inefficiencies found in traditional cryoprobe systems. Most conventional cryosurgical probe instruments operate with liquid nitrogen or other liquefied gas as the cooling medium. The LN₂ is introduced into the freezing zone of the probe through an inlet tube (which is usually the innermost tube of three concentric tubes). The inlet tube extends into an expansive chamber at the closed probe tip end but terminates a distance from the tip. The LN₂ immediately and rapidly vaporizes and undergoes over a one hundred-fold increase in volume. As the liquid vaporizes, it absorbs heat from the probe tip to lower its temperature, theoretically to the normal boiling point of LN₂ (about −196° C.). However, in actual practice as liquid nitrogen boils, a thin layer of nitrogen gas inevitably forms on the inner surface of the closed probe tip end. This gas layer has a high thermal resistance and acts to insulate the probe tip freezing zone such that the outside probe tip temperature does not usually fall below about −160° C. This effect is known as the Liedenfrost effect. Other inefficiencies found in traditional cryoprobe systems include vapor lock. Vapor lock occurs when the back pressures produced by the boiling LN₂ reduce the LN₂ flow into the freezing zone, thereby further reducing the efficiency of the probe tip cool. Rubinsky sub-cools the LN₂ as a way to overcome these inefficiencies.

In order to address inefficiencies found in traditional cryoprobe systems, Littrup takes a different approach than Rubinsky. Littrup pressurizes the liquid nitrogen to near critical pressures along the phase diagram to pressures of about 494 psi (nearly 33.5 atmospheres) to overcome the Liedenfrost effect and back pressure. The Littrup system uses a cryotherapy probe with a shaft having a closed distal end adapted to insertion into a body and having a hollow zone within the shaft. A thermally isolated inlet capillary is provided in fluid communication with the hollow zone for providing a flow of liquid towards the hollow zone. An outlet capillary is provided in fluid communication with the hollow zone for providing a flow of liquid away from the hollow zone. A vacuum jacket is adapted to provide thermal insulation of the inlet and outlet capillaries within the shaft. The Littrup device requires two tubes thermally isolated from one another disposed within the shaft of the probe. Working pressures in the Littrup device range from 420 psi to 508 psi (29-35 bars) of pressure. The high pressure required in Littrup necessitate the use of expensive materials and fittings to maintain the cryogen at these pressures and prevent system failure.

To date, the problems inherent in liquid nitrogen systems have led to the art to avoid them in favor of gaseous argon systems. What is needed is a cryoprobe system that can utilize liquid nitrogen in a low pressure, low cost and efficient manner.

### SUMMARY

The devices and methods described below provide for use of liquid nitrogen in cryoablation systems while minimizing the amount of cryogen used during cryosurgical procedures. The system uses cryoprobes of coaxial structure, and is supplied with cryogen from a dewar of liquid nitrogen. The system includes various enhancements to avoid heat transfer from the liquid nitrogen to the system components, and as a result permits use of very low-pressure nitrogen, and, vice-versa, the use of low pressure nitrogen permits use of the various enhancements (which could not be used in a high pressure system). The result is a system that provides sufficient cooling power to effectively ablate lesions, tumors and masses within the breast of female patients while using very little nitrogen and a compact and inexpensive system based on readily available and easy to handle liquid nitrogen.

The system includes a low-pressure liquid nitrogen supply, which preferable uses only 22.5 to 29.4 psi of pressure to provide adequate cooling power for treatment of typical breast lesions. The pressure may be provided by supplying lightly pressurized air into the dewar, by heating a small portion of the nitrogen in the dewar or with a small low pressure pump. For example, our prototype utilizes a compressor commonly used in household aquariums to pressurize the dewar.

The utilization of low pressure liquid nitrogen permits use of polymers for several components, such as the supply hose, the cryoprobe inlet tube, and various hose connectors which are typically made of metal, so that the system is much more efficient and uses very little liquid nitrogen. Additionally, because the liquid nitrogen is lightly pressurized, the boiling point remains low, and the liquid temperature also remains low compared with higher pressure systems.

### BRIEF DESCRIPTION OF THE DRAWINGS

FIG. 1 illustrates a cryosurgical system which uses liquid nitrogen as a cryogen.

FIG. 2 illustrates a handle portion and supply hose.

FIG. 3 illustrates a supply hose modified to enhance operation of the system of FIG. 1.

FIG. 4 illustrates a sectional view of the dewar.

FIG. 5 illustrates a cryosurgical system with a dewar and a compressor to pressurize the cryogen disposed within the housing of the control system.

FIG. 6 illustrates the control system interface of the cryosurgical system.

FIG. 7 illustrates a cryosurgical system which uses liquid nitrogen as a cryogen and a small heater in the cryogen source to pressurize the cryogen.

FIG. 8 illustrates a cryosurgical system which uses liquid nitrogen as a cryogen and a pump for driving cryogen flow.

FIG. 9 illustrates a cryosurgical system without control valve using a compressor to regulate cryogen flow.

### DETAILED DESCRIPTION OF THE INVENTIONS

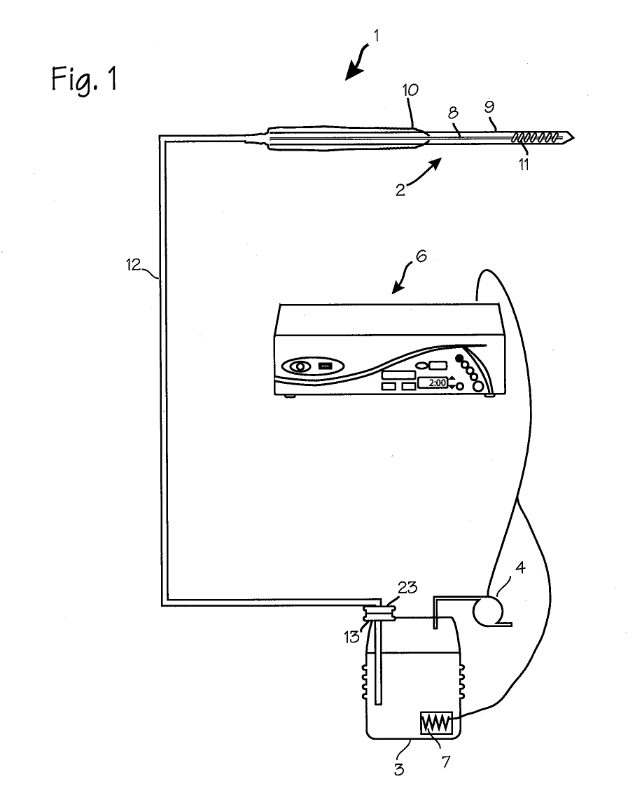

FIG. 1 illustrates a cryosurgical system which uses liquid nitrogen as a cryogen. The cryosurgical system 1 comprises cryoprobe 2, a cryogen source 3, pressurization pump 4, and a control system 6 for controlling the control valve. The system may also be provided with a cryogen source heater 7 placed in thermal communication with the cryogen source. The desired flow of cryogen from the dewar to the cryoprobe is induced in this embodiment by pressurizing the cryogen source with air delivered by the pressurized pump. The cryosurgical system 1 may be adapted to accommodate multiple cryoprobes with the addition of appropriate manifolds, and the control system may be computer-based or otherwise operable to automatically control the pressure and flow rate and other system components to effect the cooling profiles for desired cryosurgeries.

The cryogenic system 1 is arranged without a control valve in fluid communication with the fluid pathway. The necessary cryogen flow rate of the cryogen may be adjusted by regulating the pressure in the cryogen source 3 using a compressor 4. Valves act as heat sinks and are sources of cryogen leaks. Use of control valves in the fluid pathway can result in over 30% cryogen loss. Reducing or elimination the number of valves in the system 1 results in more efficient use of cryogen. The control system operably controls the compressor 4 to increase pressure in the cryogen source 3 when a higher flow rate is desired in the probe. When a higher probe temperature is desired by the user, the compressor 4 is slowed or stopped by the control system causing reduced pressure in the cryogen source 3 and reduced cryogen flow to the probe which results in a higher temperature.

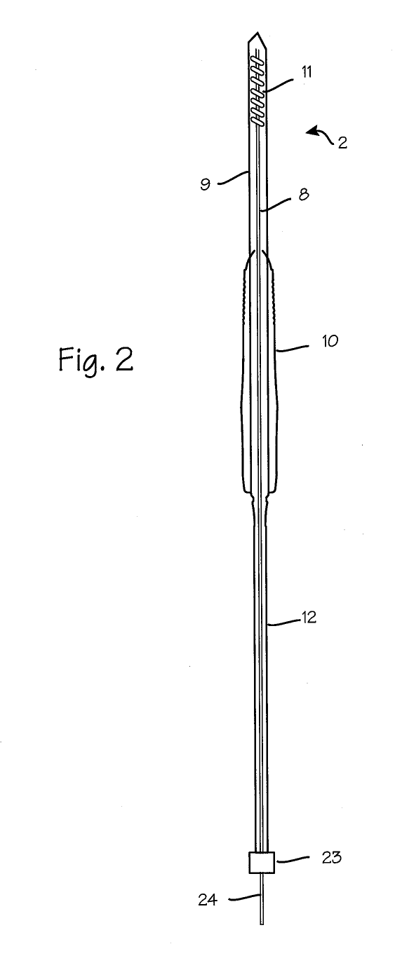

The cryoprobe 2 comprises an inlet tube 8, a closed-ended outer tube 9, and a handle portion 10. The inlet tube 8 comprises a small diameter tube, and the outer tube comprises a closed end tube, disposed coaxially about the inlet tube. The inlet tube is preferably a rigid tube with low thermal conductivity, such as polyetheretherketone (PEEK, which is well know for its high temperature performance), fluorinated ethylene propylene (FEP) or polytetrafluoroethylene. The cryoprobe preferably includes the flow-directing coil 11 or baffle disposed coaxially between the inlet tube and the outer tube at the distal end of the cryoprobe. The coil serves to direct flow onto the inner surface of the outer tube, thereby enhancing heat transfer from the outer tube that the cryogen fluid stream. The cryoprobe is describe in detail in our co-pending application, DeLonzor, et al., Cryoprobe For Low Pressure Systems, U.S. patent application Ser. No. 11/318,142 filed Dec. 23, 2005, the entirety of which is hereby incorporated by reference. The cryoprobe is supplied with cryogen from the cryogen source 3 or dewar through a supply hose 12 and the dewar outlet fitting 13. The fluid pathway of the cryogen which includes the inlet tube, the inner tube and the dip tube is devoid of high-pressure fittings or substantially metallic fittings. The handle portion 10 and supply hose 12 as shown in FIG. 2 may be integrally structured. A fluid pathway, including the inlet tube, the inner tube and the dip tubem may be manufactured from a single, continuous and uninterrupted tube devoid of intervening fittings. A single coupling disposed about the proximal end of the supply hose 12 is used to couple the supply hose to the cryogen source. The reduction and elimination of fittings result in a more efficient system since fitting locations are prone to cryogen leaks and act as heat sinks. The handle portion 10 and the outer jacket of the supply hose can be a single structure. When used in the current system, with low-pressure liquid nitrogen, the cryoprobe having an inlet tube of about 1 mm inner diameter and about 1.6 mm outer diameter, and an outer tube with about 2.4 mm inner diameter and about 2.7 mm outer diameter works well. The probes outer diameters may range from about 4 mm to about 1.5 mm.

The cryogen source 3 is preferably a dewar of liquid nitrogen. The dewar may comprise a material of low thermal conductivity, and is preferably fitted with a low pressure relief valve set to lift at about 65 to 80 psi. The dewar is lightly pressurized, to the typical operating pressures in the range of about 22.5 to 29.4 psi (1.5 to 2 bar) over ambient pressure, with air or other suitable gas, through compressor 14. Other means of pressurizing the liquid nitrogen may be used, including use of a pump at the outlet of the dewar, heating a small portion of the liquid nitrogen or gaseous nitrogen in the dewar to boost pressure in the dewar of heating the liquid nitrogen at the exit of the dewar. The system is, however, capable of pressuring the dewar in the range of about 7.25 to 220.5 psi (about 0.5 to 15 bar) over ambient pressure. However, the typical operating pressure is below about 75 psi.

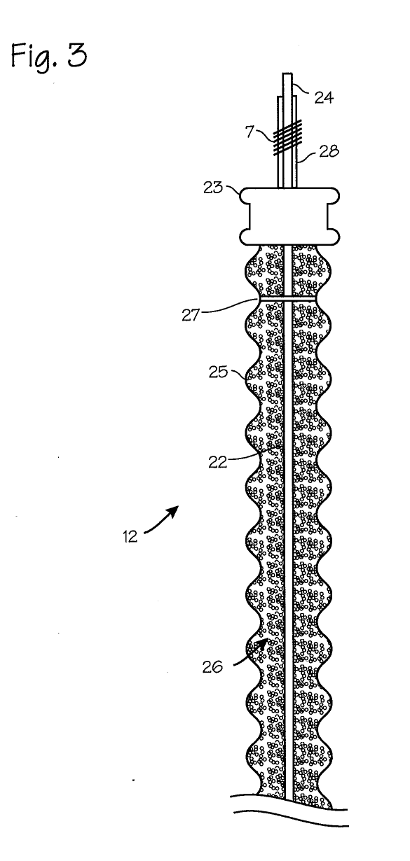

The supply hose 12, illustrated in cross section in FIG. 3, is particularly suited to use with the low-pressure liquid nitrogen system. The supply hose comprises an inner tube 22 of FEP, nylon or other thermally resistant polymer with very low thermal mass (the ability to absorb heat) (polymers typically have a low coefficient of thermal conductivity, about 0.2 to 0.3 W/mK) which remains flexible at cryogenic temperatures of the liquid nitrogen. The inner tube extends proximally beyond the supply hose coupling 23 disposed on the proximal end of the supply hose and forms a dip tube 24. The inner tube 22 of the supply hose and the dip tube can be a single tube 24 or the inlet tube 8 of the cryoprobe, the inner tube 22 of the supply hose and the dip tube can also manufactured from a single tube 24. Alternatively, the inlet tube 8 of the cryoprobe, the inner tube 22 of the supply hose and the dip tube may be bonded together without the use of high-pressure fittings. When the supply hose is coupled to the dewar, the dip tube extends into the dewar placing the dip tube in fluid communication with the cryogen. The outer tube or jacket 25 of the supply hose is manufactured from any suitable flexible material (ethylene vinyl acetate (EVA), low density polyethylene (LDPE), or nylon, for example) and may be corrugated transversely to promote omni-directional flexibility. The space between the inner tube and outer jacket is filled with aerogel beads or particles (indicated at item 26) or provided as a continuous tube of aerogel. (Aerogel refers to a synthetic amorphous silica gel foam, with a very low thermal conductivity (10⁻³ W/mK and below) with pores sizes in the range of about 5 to 100 nm.) The supply hose is preferably about 1.5-3 feet long, which provides convenient working length while minimizing cooling losses. The outer tube is preferably about 15 mm in outer diameter, while the inner tube is preferably about 1 mm in inner diameter and 1.5 mm outer diameter. Occasional spacers, in the form of washers 27 comprising materials such as polymethacrylimide closed-cell foam (PMI), may be placed along the inner tube to prevent collapse of the outer jacket and displacement of the aerogel beads. An aerogel tube may be formed by wrapping flexible aerogel blankets around the inner tube, or extruding and aerogel and binder mixture. The annular space between the inner tube and outer jacket of the supply hose may also be filled with other low thermal mass materials such as perlite powder, cotton fiber, etc., though aerogel has proven particularly effective in limiting warming of the cryogen within the supply tube while providing a supply hose that is easy to manipulate during the course of a cryosurgical procedure. An insulating layer 28 may also be disposed about the dip tube 24 to reduce temperature loss of the cryogen when flowing through the dip tube 24. Coupling 23 is provided to releasably attach the supply hose to the dewar, so that the supply hose can readily be attached and detached from the dewar without use of special tools. The couplings in the system 1 may comprise any releasable fitting structure, such as Luer fittings, bayonet fittings, large threaded fitting that are operable by hand, quick-lock fittings and the like.

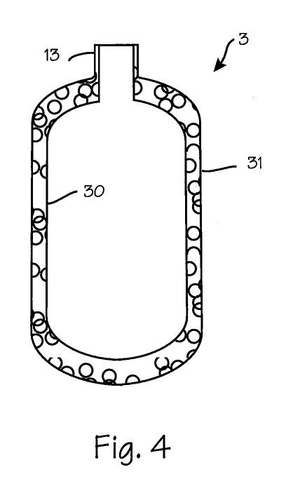

FIG. 4 illustrates a sectional view of the dewar as the cryogen source 3. The dewar comprises a liquid nitrogen vessel 30 containing liquid nitrogen surrounded by an outer housing 31. The dewar outlet fitting 13 enables the dewar to be coupled with the supply hose when the supply house coupling 23 is disposed about the fitting. The space between the vessel and outer housing is filled with low thermal mass materials such as aerogel beads, particles or a continuous tube of aerogel. The annular space between the inner vessel and outer housing of the dewar may also be filled with other low thermal mass materials such as perlite powder, cotton fiber, etc., though aerogel has proven particularly effective in limiting warming of the cryogen within the dewar during the course of a cryosurgical procedure.

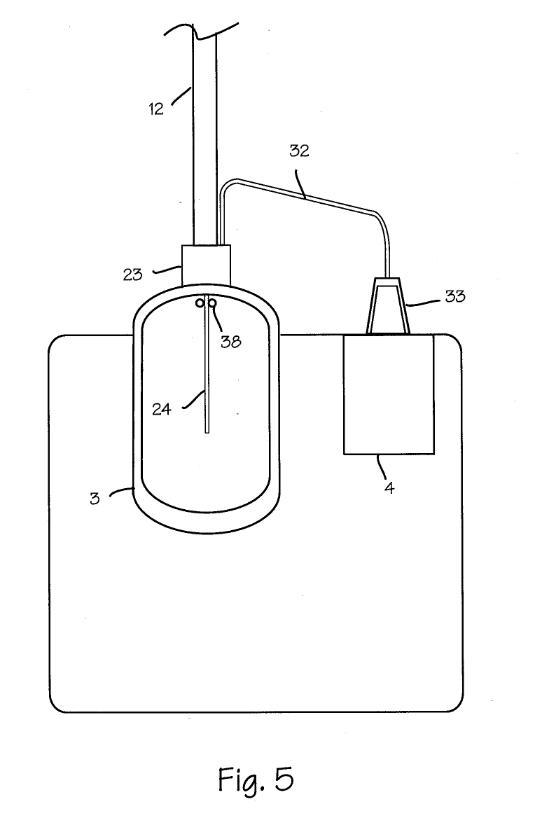

FIG. 5 illustrates a detailed sectional view of the cryogen source and the compressor 4. The compressor is placed in fluid communication with the dewar at the outlet of the dewar, through a low pressure supply tube 32 in fluid communication with the supply hose coupling 23 and the low pressure supply tube coupling 33. The compressor is operable by the control system to provide air pressure between about 5. To 15 bar of pressure to the cryoprobe. The system typically provides nitrogen between about 22.5 to 29.4 psi (1.5 to 2 bars). The dip tube 24 extends proximally beyond the proximal end of the jacket of the supply hose 12 and the supply hose coupling 23 and is disposed within the cryogen source 3 while being placed in fluid communication with the liquid in the source 3. A peristaltic valve 38 or pinch valve may be used to regulate flow of cryogen through the dip tube. The peristaltic valve 38 is disposed within the dewar and operably connected to the dip tube 24. The valve may be operably connected to a control system and flow rate may be controlled by the system.

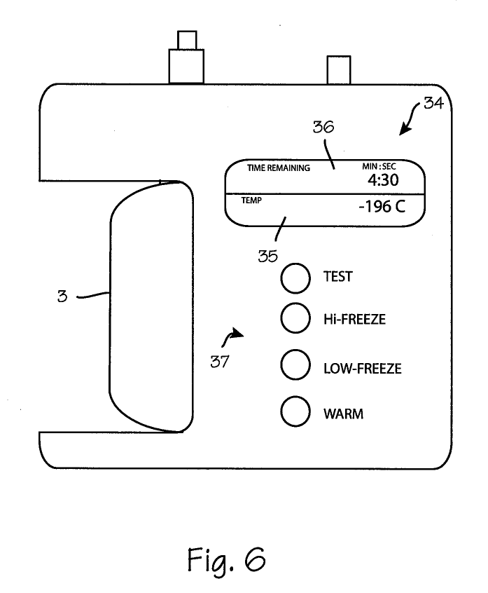

The control system interface 34 is illustrated in FIG. 6. The interface comprises a digital display or other suitable means for displaying information such as an LCD or OLED. The display contains a probe temperature indicator 35 for displaying the temperature of the probe as well as a time remaining indicator 36 for displaying the amount of freezing time available in the system. The interface further comprises cycle indicator lights 37 to indicate to the operator that the system is testing itself, performing a Hi-freeze procedure, performing a low freeze procedure, thawing the target tissue or warming the cryogen. The cycle indicators lights are operable by the control system to indicate the current status of the system. Membrane switches, or any other form of input device may be used as input buttons for the control system. The indicator lights may be replaced with any form of visual, audible, or tactile indicator capable of providing several distinct signals to the user.

In use, the cryoprobe is inserted into the body, with its distal tip within a lesion or other diseased tissue that is to be ablated, the surgeon will operate the systems through controls on the control system. The dewar may be pressurized to between about 0.5 to 15 bar (about 7.25 to 220.5 psi). Preferably, the dewar is pressurized to about 22.5 to 29.4 psi. The dewar is pressurized to provide flow to the cryoprobe at about 0.5 to 2 grams per second to effect cryoablation of the lesion. The flow of cryogen is continued as necessary to freeze the lesion to cryogenic temperatures. Preferably the operation of the system is controlled automatically via the control system, though it may be implemented manually by a surgeon, including manual operation of the pressurization means of the dewar. When used to treat lesions in the breast, the system may be operated according to the parameters described in our U.S. Pat. No. 6,789,545.

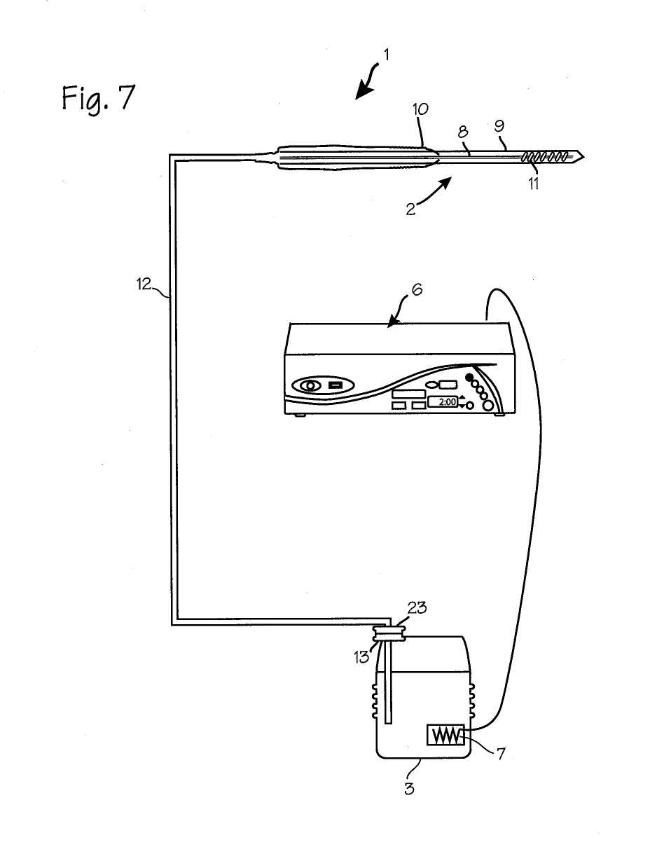

FIG. 7 illustrates a liquid nitrogen cryosurgical system which uses a heater to generate the desired pressure to drive the system. This system includes the cryoprobe 2, cryogen source 3 and control system 6 of FIG. 1. A heater 7 is provided in the dewar, and is operable to heat a small volume of the nitrogen in the dewar and thereby increase the pressure in the dewar to the desired level of 0.5 to 15 bar (7.25 to 220.5 psi) above ambient pressure. The control system can automatically control the heater with feedback from pressure sensors in the dewar. The heater 7 may be submersed in the liquid nitrogen or placed within the gas above the liquid. It may be disposed on the inside wall of the dewar or suspended within the dewar. The heater 7 may also be disposed on the dip tube. In another embodiment of the system, a heater 7 may be placed in thermal communication with the dewar by disposing a heater outside the vessel 30.

As shown in FIG. 7, the necessary cryogen flow rate may be adjusted by regulating the pressure in the dewar using the heater. The pressure in the cryogen source 3 or dewar is generated through use of the heater. The control system 6 operably controls the heater to heat the cryogen and increase pressure in the cryogen source 3 when a higher flow rate and lower temperature is desired in the probe. When a lower probe temperature is desired by the user, the heating of the cryogen is reduced or stopped by the control system 6 causing reduced pressure in the dewar and reduced cryogen flow to the probe 2.

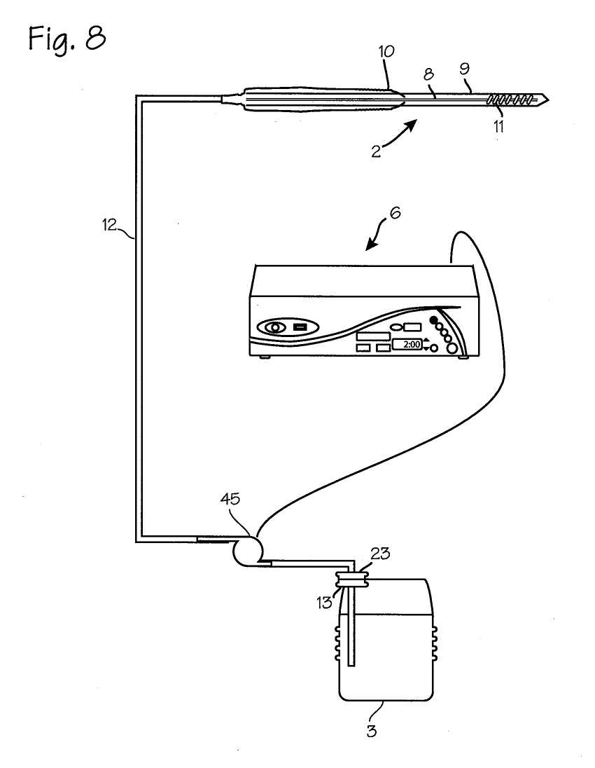

As shown in FIG. 8, the necessary pressure may also be provided with a cryogenic pump 45. In FIG. 8, a cryogenic pump is placed at the outlet of the dewar, in line with the dewar outlet hose 13, and is operable by the control system to provide liquid nitrogen at about 5. to 15 bar of pressure to the control valve and cryoprobe. The use of air, as shown in FIG. 1, and the use of the heater as shown in FIG. 7, both entail addition of heat to the dewar system, but this has proven acceptable given the additional thermal gains obtained by the various components described above. The necessary cryogen flow rate may be adjusted by regulating pump. The control system 6 operably controls the pump increase flow rate when a higher flow rate and lower temperature is desired in the probe. When a lower probe temperature is desired by the user, flow rate by the pump is reduced or stopped by the control system 6 causing reduced cryogen flow to the probe 2.

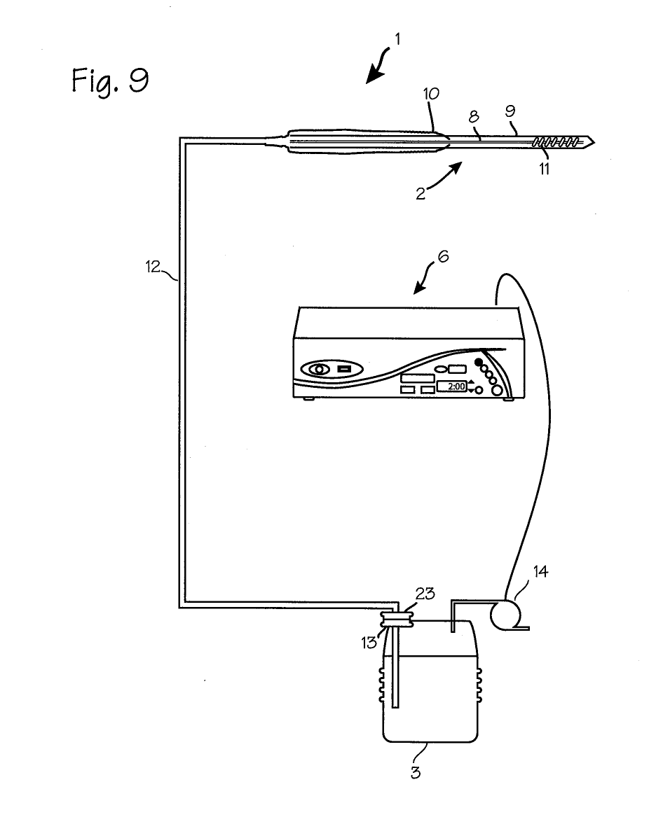

A cryogenic system without a control valve using a compressor to regulate the pressure in the dewar is illustrated in FIG. 9. As shown in FIG. 9, the necessary cryogen flow rate may be adjusted by regulating the pressure in the cryogen source 3 using a compressor 4. Valves act as heat sinks and are sources of cryogen leaks. Reducing or elimination the number of valves in the system 1 results in more efficient use of cryogen. In FIG. 9, the necessary pressure in the cryogen source 3 is provided the compressor 4. The control system 6 operably controls the compressor 4 to increase pressure in the cryogen source 3 when a higher flow rate and lower temperature is desired in the probe. When a lower probe temperature is desired by the user, the compressor 4 is slowed or stopped by the control system causing reduced pressure in the cryogen source 3 and reduced cryogen flow to the probe.

The systems described above may be employed with various liquid cryogens, though liquid nitrogen is favored for is universal availability and ease of use. Also, though system has been developed for use in treatment of breast disease, it may be employed to treat lesions elsewhere in the body. Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A cryosurgical system comprising:
a cryoprobe comprising a handle portion, a closed-ended outer tube disposed within the handle portion, an inlet tube disposed within the outer tube and a flow directing coil disposed between the inlet tube and outer tube, at the distal end of the cryoprobe, said cryoprobe having a distal end corresponding to the closed end of the outer tube which is adapted for insertion into the body of a patient and a proximal end adapted for connection to a source of cryogenic liquid;
a supply hose connecting the proximal end of the cryoprobe to a source of cryogenic liquid, said supply hose establishing a liquid flow path from the source of cryogenic liquid to the inlet tube of the cryoprobe, said liquid flow path devoid of intervening couplings;
a pressurizing means for pressuring the cryogenic liquid;
a control system operable to control the pressurizing means to provide cryogenic liquid to the cryoprobe and to pressurize the source in the range of about 0.5 to 15 bar.

2. A cryosurgical system of claim 1 wherein the supply hose and the handle portion are integrally structured.

3. A cryosurgical system of claim 1 wherein the supply hose comprises:
an inner tube comprising a polymer disposed within an outer jacket and a dip tube extending proximally beyond a proximal end of the outer jacket in fluid communication with the inner tube.

4. A cryosurgical system of claim 3 wherein the inner tube and the dip tube are a single tube.

5. A cryosurgical system of claim 3 wherein the inlet tube, the inner tube and the dip tube are a single tube.

6. A cryosurgical system of claim 3 wherein said inner tube has an inner diameter of about 1 mm and said outer jacket has a diameter of about 15 mm with a space between inner tube and outer tube being filled with aerogel.

7. A cryosurgical system of claim 3 further comprising:
means for releasably attaching the supply hose proximal end to the source of liquid cryogen, said means comprising low thermal mass polymeric fittings.

8. A cryosurgical system of claim 1 wherein the pressurizing means comprises:
a compressor operably connected to the source to pump air into the source and thereby pressurize the source to about 0.5 to 15 bar of pressure.

9. A cryosurgical system of claim 1 wherein the pressurizing means comprises:
a heater in thermal communication with the cryogen in the source, said heater being operable to heat a small volume of the cryogen and thereby pressurize the source to about 0.5 to 15 bar of pressure.

10. A cryosurgical system of claim 2 further comprising a cryogen heater disposed on the dip tube.

11. A cryosurgical system of claim 7 further comprising:
a heater in thermal communication with the cryogen in the source.

12. A cryosurgical system of claim 1 wherein the pressurizing means comprises:
a pump operably connected to the source to pump cryogen from the source to the cryoprobe at a pressure of about 0.5 to 15 bar of pressure.

13. A cryosurgical system of claim 3 wherein the handle portion and the outer jacket are a single structure.

14. A cryosurgical system of claim 1 wherein the flow path is devoid of intervening control valves.
